(19) United States
(12) Patent Application Publication
(10) Pub. No.: US 2025/0256055 A1
(43) Pub. Date: Aug. 14, 2025

(54) METHODS AND SYSTEMS FOR INDUCING SLEEP IN A SUBJECT

(71) Applicant: Teledyne Scientific & Imaging, LLC, Thousand Oaks, CA (US)

(72) Inventors: Stephen B. Simons, Raleigh, NC (US); Maria Provo, Cary, NC (US); Michael Weisend, Efland, NC (US); Patrick M. Connolly, Cary, NC (US); Renee Shimizu, Raleigh, NC (US)

(21) Appl. No.: 19/046,638

(22) Filed: Feb. 6, 2025

Related U.S. Application Data
(60) Provisional application No. 63/551,902, filed on Feb. 9, 2024.

(51) Int. Cl. A61M 21/02; A61M 21/00

(52) U.S. Cl. CPC A61M 21/02; A61M 2021/0022; A61M 2021/0044; A61M 2021/0055; A61M 2021/0072; A61M 2230/06; A61M 2230/10

(57) ABSTRACT

Methods and systems for inducing sleep in a subject. The method includes emitting, by a transducer, a first neuromodulatory signal to the subject based on a signal parameter during a first time period. A first biomarker signal of the subject is measured by a first detector a during a second time period. The second time period is subsequent to or at least partially overlaps with the first time period. The method includes determining, by a controller, a characteristic of the first biomarker signal and adjusting, by the controller, the signal parameters based on the characteristic thereby forming an adjusted parameter. Based on the adjusted parameter, the method includes performing at least one of the following: emitting, by the transducer, a second neuromodulatory signal to the subject during a third time period based on the adjusted parameter; or ceasing further emission of neuromodulatory signals based on the adjusted parameter.

### PRIORITY

The present application claims priority under 35 U.S.C. Section 119 (e) from Provisional Application 63/551,902, entitled “METHODS AND SYSTEMS FOR INDUCING SLEEP IN A SUBJECT,” filed on Feb. 9, 2024, the entire contents of which is incorporated herein by reference.

### FIELD OF USE

The present disclosure relates to methods and systems for inducing sleep in a subject.

### BACKGROUND

Sleep is a necessary activity for humans along with other animals. Sleep may be characterized by a period of inhibited sensory activity, reduced muscle activity especially of the voluntary muscles, and general reduced interactions with the surroundings. Several important physiological functions occur during sleep including, without limitation, restoration processes involved with the metabolism, muscle and bone growth and repair, wound healing, and memory consolidation in the brain. Lack of sleep, or restful sleep, may reduce or interfere with these processes and has also been correlated with cardiovascular disease and diabetes. Conditions that may inhibit sleep or restful sleep may include, without limitation, sleep apnea, bruxism, nocturia, restless leg syndrome, and insomnia.

Insomnia may relate to either difficulty in falling asleep or waking during normal sleep. Some form of insomnia has been shown to exist in about 10% to about 30% of the general population. However, incidence of insomnia in older adults (age 65 and older) may be much greater and may be in the range of about 50% to 60% of that population. Higher levels of incidence of insomnia in that age group may be found. According to some investigations, around 60 million people suffer from some form of insomnia in the U.S. alone.

Methods of addressing insomnia may include the use of certain types of transcranial stimulation to induce sleep. However, there are challenges associated with using transcranial stimulation effectively for this purpose.

### SUMMARY

One non-limiting aspect according to the present disclosure is directed to a method for inducing sleep in a subject. The method comprises emitting, by a transducer, a first neuromodulatory signal to the subject based on a signal parameter during a first time period. A first biomarker signal of the subject is measured by a first detector during a second time period. The second time period is subsequent to, or at least partially overlaps with, the first time period. The method comprises determining, by a controller, a characteristic of the first biomarker signal and adjusting, by the controller, the signal parameters based on the characteristic thereby forming an adjusted parameter. The method further comprises, based on the adjusted parameter, performing at least one of the following: emitting, by the transducer, a second neuromodulatory signal to the subject during a third time period based on the adjusted parameter; or ceasing further emission of neuromodulatory signals based on the adjusted parameter.

Another non-limiting aspect of the present disclosure is directed to a system for inducing sleep in a subject. The system comprises a transducer, a detector, and a controller. The transducer is capable to emit a first neuromodulatory signal to the subject based on a signal parameter during a first time period. The detector is capable to measure a biomarker signal of the subject during a second time period. The second time period is subsequent to or at least partially overlaps with the first time period. The controller is in signal communication with the transducer and the detector. The controller is capable to determine a characteristic of the biomarker signal and adjust the signal parameter based on the at least one characteristic thereby forming an adjusted parameter. The controller is capable to perform at least one of the following based on the adjusted parameter: cause the transducer to emit a second neuromodulatory signal to the subject during a third time period based on the adjusted parameter; or cease further emission of neuromodulatory signals based on the adjusted parameter.

It will be understood that the inventions disclosed and described in this specification are not limited to the aspects summarized in this Summary. The reader will appreciate the foregoing details, as well as others, upon considering the following detailed description of various non-limiting and non-exhaustive aspects according to this specification.

### BRIEF DESCRIPTION OF THE DRAWINGS

The features and advantages of the examples presented herein, and the manner of attaining them, will become more apparent, and the examples will be better understood, by reference to the following description taken in conjunction with the accompanying drawings, wherein:

FIG. 1 is a schematic representation of a non-limiting embodiment of a system for inducing sleep according to the present disclosure; and

FIG. 2 illustrates a non-limiting method for inducing sleep according to the present disclosure.

The exemplifications set out herein illustrate certain non-limiting embodiments, in one form, and such exemplifications are not to be construed as limiting the scope of the appended claims and the invention in any manner.

### DETAILED DESCRIPTION OF NON-LIMITING EMBODIMENTS

Various examples are described and illustrated herein to provide an overall understanding of the structure, function, and use of the disclosed methods and system. The various examples described and illustrated herein are non-limiting and non-exhaustive. Thus, the invention is not limited by the description of the various non-limiting and non-exhaustive examples disclosed herein. Features and characteristics illustrated and/or described in connection with various examples herein may be combined with features and characteristics of other examples herein. Such modifications and variations are intended to be included within the scope of the present disclosure. The various non-limiting embodiments disclosed and described in the present disclosure can comprise, consist of, or consist essentially of the features and characteristics as variously described herein.

Any references herein to “various non-limiting embodiments”, “some non-limiting embodiments”, “certain non-limiting embodiments”, “one non-limiting embodiment”, “a non-limiting embodiment”, “an embodiment”, “one embodiment”, or like phrases mean that a particular feature, structure, act, or characteristic described in connection with the example is included in at least one embodiment. Thus, appearances of the phrases “various non-limiting embodiments”, “some non-limiting embodiments”, “certain non-limiting embodiments”, “one non-limiting embodiment”, “a non-limiting embodiment”, “an embodiment”, “one embodiment”, or like phrases in the specification do not necessarily refer to the same non-limiting embodiment. Furthermore, the particular described features, structures, or characteristics may be combined in any suitable manner in one or more non-limiting embodiments. Thus, the particular features, structures, or characteristics illustrated or described in connection with one non-limiting embodiment may be combined, in whole or in part, with the features, structures, or characteristics of one or more other non-limiting embodiments without limitation. Such modifications and variations are intended to be included within the scope of the present non-limiting embodiments.

As used herein, “at least one of” a list of elements means one of the elements or any combination of two or more of the listed elements. As an example “at least one of A, B, and C” means A only; B only; C only; A and B; A and C; B and C; and A, B, and C.

There are various known treatment methods for inducing sleep in a subject. However, each subject may respond differently to a particular treatment method, and a particular subject may respond differently between treatment methods. The present disclosure provides methods for inducing sleep that can dynamically adjust the treatment based on how the subject responds to the treatment, thereby tailoring the treatment to the subject at the time of treatment. The methods and systems according to the present disclosure can reduce the total dose required, the total time required, and/or increase efficacy of the treatment. For example, a non-limiting embodiment of a method according to the present disclosure comprises emitting, by a transducer, a first neuromodulatory signal to the subject based on a signal parameter during a first time period. A first biomarker signal of the subject can be measured by a first detector during a second time period, wherein the second time period is subsequent to or at least partially overlaps with the first time period. The method can comprise determining, by a controller, a characteristic of the first biomarker signal and adjusting, by the controller, the neuromodulatory signal parameters based on the characteristic, thereby forming an adjusted parameter. The method can further comprise, based on the adjusted parameter, performing at least one of the following: emitting, by the transducer, a second neuromodulatory signal to the subject during a third time period based on the adjusted parameter; or ceasing further emission of neuromodulatory signals based on the adjusted parameter.

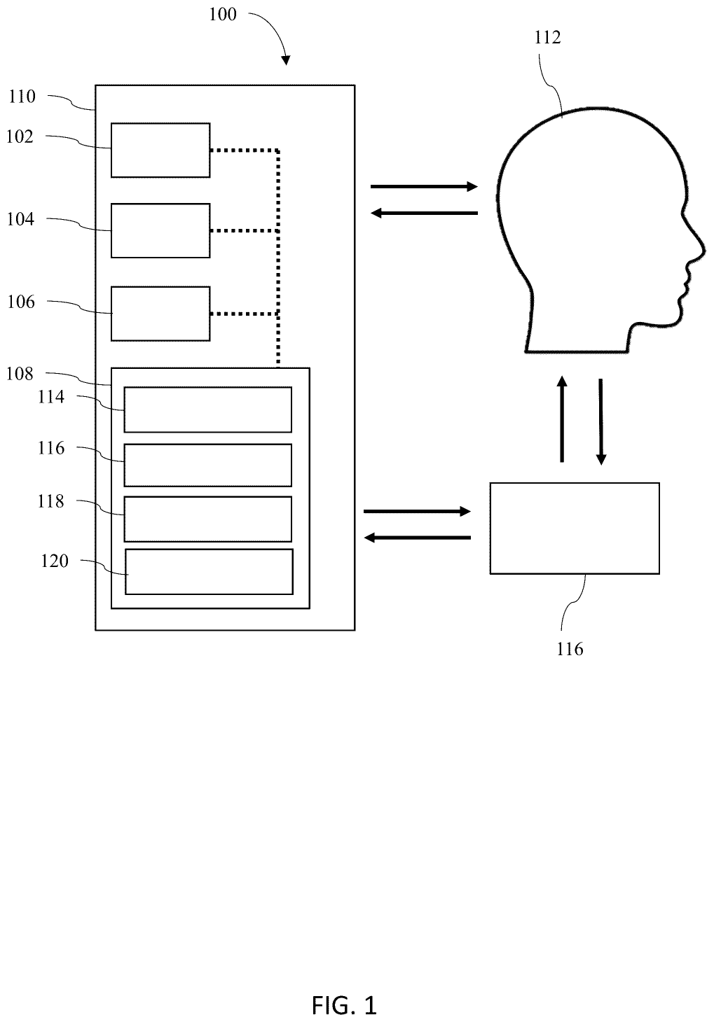

Referring to FIG. 1, a system 100 for inducing sleep according to the present disclosure is provided. The system 100 can be capable of non-invasive treatment of insomnia in a subject by inducing sleep in the subject. The system 100 comprises a transducer 102, a detector 104, and a controller 106 in signal communication with the transducer 102 and the detector 104.

In certain non-limiting embodiments, the system 100 can comprise a housing 110 capable of being worn by a subject while awake and/or asleep. The transducer 102 and the detector 104 can be disposed within or on the housing 110. The housing 110 can comprise various components, such as, for example, a hat, a headband, and/or other head-worn device. The housing 110 may comprise, for example, a cloth and/or elastic material, which may be positioned on a forehead of a subject 112 and wrapped around the circumference of a head of the subject 112. In certain embodiment, the housing 110 is capable of conforming to a shape of a head of a subject. The housing 110 may comprise other components as necessary so that is may be attached to and/or worn by the subject 112.

The system 100 can comprise a single transducer 102 or at least two transducers 102, such as, for example, at least three, at least four, at least six, at least eight, or at least ten transducers 102. The transducer 102 can be capable to emit a neuromodulatory signal to a subject 112 based on a signal parameter 114. The transducer 102 can comprise at least one hardware element selected from the group consisting of an electrode (e.g., a hydrogel electrode), an ultrasonic transducer, a magnetic field transducer, a visual transducer (e.g., an LED and/or a visual display), and an acoustic transducer (e.g., a speaker). In various non-limiting embodiments, the transducer 102 can comprise at least one hardware element selected from the group consisting of an electrode, an ultrasonic transducer, and a magnetic field transducer. The neuromodulatory signal can comprise at least one form of non-invasive neuromodulation selected from the group consisting of an electrical stimulation, an ultrasound stimulation, a magnetic field stimulation, a visual stimulation, and an acoustic stimulation. In various non-limiting embodiments, the neuromodulatory signal can comprise at least one form of non-invasive transcranial neuromodulation selected from the group consisting of an electrical stimulation, an ultrasound stimulation, and a magnetic field stimulation. The neuromodulatory signal can provide stimulation to the subject 112 that may induce sleep.

For example, the transducer 102 can comprise an electrode capable of providing electrical stimulation comprising an electrical current by direct contact with skin of the head of the subject 112; the transducer 102 can comprise an ultrasonic transducer capable of providing ultrasound stimulation through the skin of the head of the subject 112; the transducer 102 can comprise an electromagnetic field transducer capable of providing magnetic field stimulation to the head of the subject 112; the transducer 102 can comprise a visual transducer capable of providing visual stimulation (e.g., visual light) through the visual system (e.g., an eye, eyes) of the subject 112; and/or the transducer 102 can comprise an acoustic transducer capable of providing acoustic stimulation (e.g., through speakers/headphones) to the auditory system (e.g., ear drum) of the subject 112. In various non-limiting embodiments in which the transducer 102 comprises an electrode capable of providing electrical stimulation, the electrical stimulation can comprise a waveform similar to that generated by the brain of the subject 112 during sleep, such as, for example, during non-REM stage 3 sleep.

As described above, the neuromodulatory signal can be emitted based on the signal parameter 114. The signal parameter 114 can comprise various instructions on how the transducer 102 can be configured and/or operate during a treatment. The signal parameter 114 can be initialized by the subject 112 manually, by a prior treatment session, and/or via a database of parameters. The signal parameter 114 can comprise one or more parameters selected from the group consisting of a time of stimulation, an intensity of stimulation, a frequency of stimulation, a frequency of application of stimulation, a rate of application of stimulation, and a state parameter (e.g., on/off). In various non-limiting embodiments, the signal parameter 114 comprises a time of stimulation and an intensity of stimulation. The signal parameter 114 may be stored, for example, in a memory 108 of the system 100, on the cloud, on a server in signal communication with the system 100, or at another location in signal communication with the system 100. The memory 108 can comprise, for example, primary storage (e.g., main memory that is directly accessible by a processor, such as RAM, ROM processor registers or processor cache); secondary storage (e.g., SSDs or HDDs that are not directly accessible by a processor); and/or off-line storage.

The system 100 can comprise a single detector 104 or at least two detectors 104, such as, for example, at least three, at least four, at least six, at least eight, or at least ten detectors 104. The detector 104 can be capable to measure a biomarker signal of the subject 112. For example, the detector 104 can comprise at least one hardware element selected from the group consisting of an electroencephalogram (EEG) detector (e.g., an EEG including silver/silver chloride electrodes), a magnetoencephalography (MEG) detector, and a heart monitor. The biomarker signal comprises at least one non-invasively measured signal selected from the group consisting of an EEG signal, a MEG signal, and a heart rate. The biomarker signal can provide information regarding brain activity and/or heart activity of the subject 112, which can be used to determine a sleep state of the subject 112. The biomarker signal can be used for determining a change in the subject 112 evoked by the stimulation.

For example, an EEG detector can be capable of measuring an EEG signal of the brain of the subject 112 by direct contact with skin of the head of the subject 112; an MEG detector can be capable of measuring a MEG signal of the brain of the subject 112 while disposed proximal to the subject 112; and a heart rate monitor can measure a heart rate of the subject 112 through various means, such as, for example, through electrocardiography, photoelectric pulse wave, blood pressure measurement, and/or phonocardiography techniques.

In various non-limiting embodiments, the transducer 102 comprises an electrode and the detector 104 comprises an EEG detector. In certain embodiments comprising an electrode and an EEG detector, the system 100 can further comprise a visual transducer and/or an acoustic transducer.

The controller 106 can be in signal communication with the transducer 102 and the detector 104. For example, the controller 106 can be hardwired to and/or in wireless communication (e.g., Bluetooth™, Bluetooth Low Energy™, WiFi, or other wireless connection) with the transducer 102 and the detector 104. In certain embodiments, the controller 106 may be disposed within or on the housing 110 of the system 100, within or on a secondary device 116 (e.g., a mobile disposed device) of the system 100, or a combination thereof.

As used herein, the term “controller” may refer to, for example, hardwired circuitry, programmable circuitry (e.g., a computer processor comprising one or more individual instruction processing cores, a processing unit, a processor, a microcontroller, a microcontroller unit, a controller, a digital signal processor (DSP), a programmable logic device (PLD), a programmable logic array (PLA), or a FPGA), state machine circuitry, firmware that stores instructions executed by programmable circuitry, or any combination thereof. The controller 106 may be embodied, collectively or individually, as circuitry that forms part of a larger system, for example, an IC, an ASIC, a SoC, a desktop computer, a laptop computer, a tablet computer, a server, or a smart phone. Accordingly, as used herein, a “controller” can comprise electrical circuitry having at least one discrete electrical circuit, electrical circuitry having at least one IC, electrical circuitry having at least one application-specific IC, electrical circuitry forming a general-purpose computing device configured by a computer program (e.g., a general-purpose computer configured by a computer program that at least partially carries out processes and/or devices described herein, or a microprocessor configured by a computer program that at least partially carries out processes and/or devices described herein), electrical circuitry forming a memory device (e.g., forms of RAM), and/or electrical circuitry forming a communications device (e.g., a modem, a communications switch, or optical-electrical equipment). The subject matter described herein may be implemented in an analog fashion, a digital fashion, or some combination thereof.

The secondary device 116, if present, can comprise a desktop computer, a laptop computer, a tablet computer, a server, a smart phone, an application specific computing device, and/or other hardware device. The secondary device 116 can comprise an interface capable to receive input from a user to direct the functions of the secondary device 116 and/or the system 100. The interface can comprise a button, a key pad, a touch-pad, a microphone (e.g., to receive user voice commands), and/or other input device. In various non-limiting embodiments, the secondary device 116 can provide output information to the subject 112, such as, for example, information related to the status or function of the secondary device 116 and/or of the system 100 through a visual display, LED, audio, and/or other output device. In various non-limiting embodiments, the secondary device 116 can comprise an acoustic transducer and/or a visual transducer.

The controller 106 may be capable of performing various functions. For example, the controller 106 can operate the transducer 102 and the detector 104. The controller 106 can determine a characteristic of the biomarker signal received by the detector 104. The characteristic of the biomarker signal can comprises at least one characteristic selected from the group consisting of a heart rate, a synchronous measurement, an amplitude based measurement, a delta band spectral power, a phase locking value, and a means squared coherence. The characteristic of the biomarker signal can provide information regarding brain activity and/or heart activity of the subject 112.

The controller 106 can adjust a value of the signal parameter 114 based on the characteristic. The adjustment can be based on the subject's 112 response to the neuromodulatory signal and/or sleep state, which can be determined by a comparison of the characteristic of the biomarker signal to a threshold 118. The threshold 118 can be based on a predetermined value, a previous characteristic of the subject 112, or a combination thereof. The controller 106 can store the adjusted parameter in memory by overwriting the signal parameter 114 or creating a new adjusted parameter 120 in a separate location in memory 108. Adjusting the signal parameter 114 can tailor the treatment to the subject 112.

Based on the adjusted parameter, the controller 106 can determine whether or not to continue treatment of the subject 112. For example, if the characteristic achieves or exceeds a predetermined threshold, the adjusted parameter may be a state off parameter indicating that further treatment of the subject 112 should be ceased (e.g., no further neuromodulatory signals), which may be based on the subject 112 having achieved a desired degree of sleep. In various non-limiting embodiments, the controller 106 can determine an aggregate parameter based on an aggregated sum of the neuromodulatory signals emitted to the subject 112 during the treatment and compare the aggregate parameter to a limit threshold. Based on the comparison, the controller 106 can stop or continue treatment. The aggregate parameter can comprises at least one parameter selected from the group consisting of total emission time and total emission power.

If treatment is continued, the controller 106 can cause the transducer 102 to emit another neuromodulatory signal to the subject 112 based on the adjusted parameter. Utilizing the adjusted parameter can induce sleep and reduce the total dose of neuromodulatory signals applied to the subject during the treatment, reduce total treatment time, and/or increase efficacy of the treatment as the treatment can be dynamically adjusted to the biological response(s) of the particular subject 112 to the current treatment.

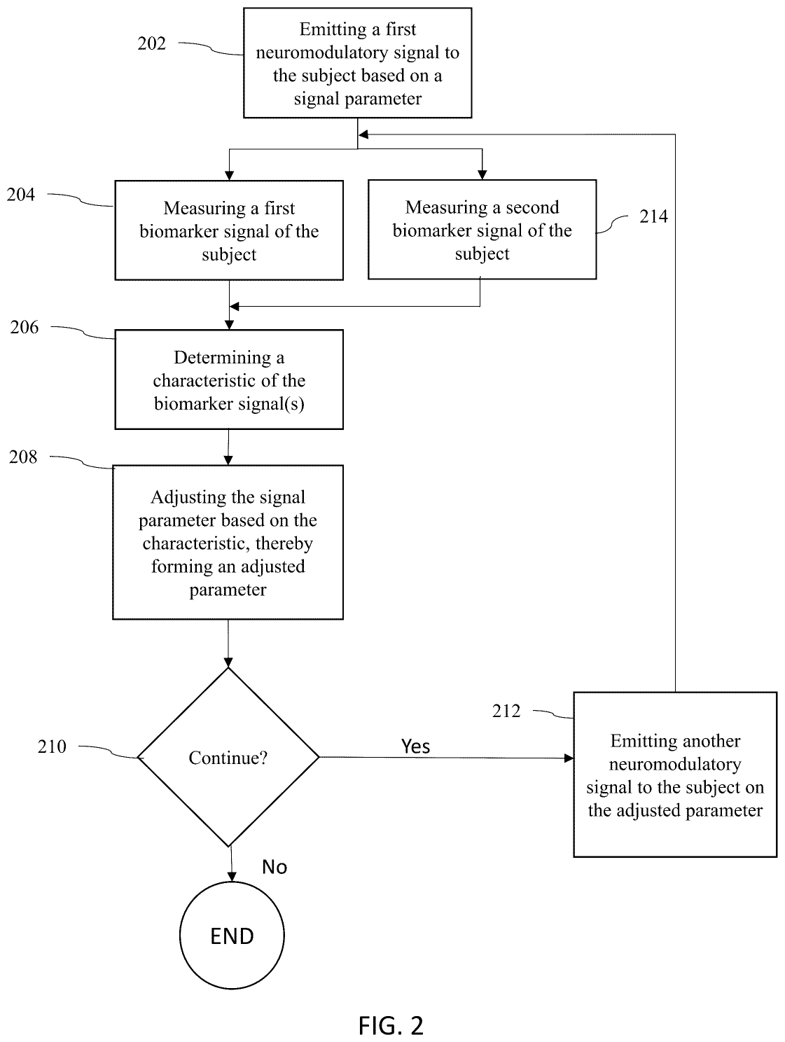

Referring to FIG. 2, an embodiment of a method for inducing sleep is provided. In various non-limiting embodiments, the method can be a closed-loop feedback method. For ease of clarity, the method in FIG. 2 is described with respect to the system in FIG. 1, although is not limited thereto. For example, the method can be performed using a system similar to that as described in U.S. patent application Ser. No. 17/160,442, which is hereby incorporated by reference, and/or other device suitable for inducing sleep.

The method can comprise emitting, by the transducer 102, a first neuromodulatory signal to the subject 112 based on a signal parameter during a first time period at step 202. Step 202 can comprise emitting a single type of neuromodulatory signal or multiple types of neuromodulatory signals, depending on the application.

The method can further comprise measuring, by the detector 104, a first biomarker signal of the subject 112 during a second time period at step 202. The second time period is subsequent to or at least partially overlaps with the first time period. In other words, the second time period may start simultaneously with or during the first time period and end prior to, simultaneously with, or after the first time period. The second time period may not be prior to the first time period. The first time period can be in a range of 4 seconds to 8 seconds. The second time period can be in a range of 1 second to 60 seconds.

The measurement of the biomarker signal can be used to determine how the subject 112 is responding to the neuromodulatory signal. In various non-limiting embodiments, some methods of emitting the neuromodulatory signal and measurement of the biomarker signal can interfere with one another and, therefore, the second time period can be subsequent to the first time period and may not overlap with the first time period. In certain non-limiting embodiments comprising an EEG electrode and an EEG detector, the second time period can be subsequent to the first time period and may not overlap with the first time period. In various non-limiting embodiments, some methods of emitting the neuromodulatory signal and measurement of the biomarker signal may not significantly interfere with one another and, therefore, the second time period can at least partially overlap with the first time period. In certain non-limiting embodiments comprising an ultrasound electrode and an EEG detector, the second time period can at least partially overlap with the first time period.

In various non-limiting embodiments, the method can comprise measuring, by a second detector, a second biomarker signal of the subject 112 during a fourth time period at step 214. The fourth time period can be substantially the same as the second time period. In certain non-limiting embodiments, the first biomarker signal can be related to a first portion of a brain of the subject 112 and the second biomarker signal can be related to a second portion of the brain of the subject 112 different than the first portion.

In certain non-limiting embodiments, the controller 106 can pre-process, clean, and/or filter the biomarker signals. For example, the controller 106 can apply a bandpass filter, a mean subtraction, a segmentation algorithm, or other algorithm to the biomarker signals depending on the application.

Regardless of how many biomarker signals are measured, the method can comprise determining, by the controller 106, a characteristic of the biomarker signal(s) at step 206. The characteristic can provide information regarding the subject's 112 brain activity and/or heart activity, which can be used for determining a sleep state of the subject 112. In various non-limiting embodiments, where at least two biomarker signals are measured, step 206 can comprise comparing the first biomarker signal and the second biomarker signal to determine a state of synchrony of the brain of the subject 112.

The method can comprise adjusting, by the controller 106, the signal parameters based on the characteristic thereby forming an adjusted parameter at step 208. The adjusted parameter can be stored in memory. The adjustment can tailor the treatment to the subject 112.

The method can determine if the treatment should continue at step 210 based on the adjusted parameter. For example, if the characteristic achieves or exceeds a predetermined threshold, the adjusted parameter may be a state off parameter indicating that further treatment of the subject 112 should be ceased (e.g., no further neuromodulatory signals are applied), which may be based on determining that the subject 112 has achieved a desired degree of sleep based on the characteristic. In various non-limiting embodiments, step 210 can comprise comparing the state of synchrony to a predetermined threshold stored in memory and determining if the state of synchrony meets or exceeds the predetermined threshold. For example, the controller 106 can be capable to determine if the state of synchrony changed from the previous measurement by a certain threshold and/or if the state of synchrony achieved a certain absolute value.

In various non-limiting embodiments, the controller 106 can determine an aggregate parameter based on an aggregated sum of the neuromodulatory signals emitted to the subject 112 during the treatment and compare the aggregate parameter to a limit threshold. Based on the comparison, if the aggregate parameter meets or exceeds the threshold, further treatment, including further emission of neuromodulatory signals to the subject 112, may be ceased.

If it is determined at step 210 that application of treatment should cease, no further neuromodulatory signals are emitted to the subject 112 during the treatment and the course of treatment ceases. Subsequently, a new course of treatment can be commenced, if desired.

If it is determined at step 210 that application of treatment should continue, the transducer 102 emits another neuromodulatory signal to the subject 112 during a third time period based on the adjusted parameter at step 212. The third time period can be subsequent to the second time period. After step 212, the method can comprise returning to step 204 and, optionally, step 214 to measure the biomarker signal(s) of the subject 112 and continue conducting the method as applicable.

Various aspects of certain non-limiting embodiments of an invention according to the present disclosure include, but are not limited to, the aspects listed in the following numbered clauses.
Clause 1. A method for inducing sleep in a subject, the method comprising: emitting, by a transducer, a first neuromodulatory signal to the subject based on a signal parameter during a first time period; measuring, by a first detector, a first biomarker signal of the subject during a second time period, wherein the second time period is subsequent to or at least partially overlaps with the first time period; determining, by a controller, a characteristic of the first biomarker signal; and adjusting, by the controller, the signal parameters based on the characteristic thereby forming an adjusted parameter; and based on the adjusted parameter, performing at least one of the following: emitting, by the transducer, a second neuromodulatory signal to the subject during a third time period based on the adjusted parameter; or ceasing further emission of neuromodulatory signals based on the adjusted parameter.
Clause 2. The method of clause 1, comprising emitting, by the transducer, the second neuromodulatory signal to the subject during the third time period based on the at least one adjusted parameter.
Clause 3. The method of any of clauses 1-2, wherein the first neuromodulatory signal comprises at least one form of non-invasive neuromodulation selected from the group consisting of an electrical stimulation, ultrasound stimulation a magnetic field stimulation, an ultrasound stimulation, a visual stimulation, and acoustic stimulation.
Clause 4. The method of any of clauses 1-3, wherein the signal parameter comprises at least one parameter selected from the group consisting of time of stimulation, intensity of stimulation, frequency of stimulation, frequency of application of stimulation, rate of application of stimulation, and a state parameter.
Clause 5. The method of any of clauses 1-4, wherein the first time period is in a range of 4 seconds to 8 seconds and the second time period is in a range of 1 second to 60 seconds.
Clause 6. The method of any one of clauses 1-5, wherein the characteristic of the first biomarker signal comprises at least one characteristic selected from the group consisting of a heart rate, a synchronous measurement, an amplitude based measurement, a delta band spectral power, a phase locking value, and a means squared coherence.
Clause 7. The method of any one of clauses 1-6, wherein the first biomarker signal is related to a first portion of a brain of the subject and the method further comprises: measuring, by a second detector, a second biomarker signal of the subject during the second time period, wherein the second biomarker signal is related to a second portion of the brain of the subject different than the first portion; and comparing the first biomarker signal and the second biomarker signal to determine a state of synchrony, wherein the characteristic comprises the state of synchrony.
Clause 8. The method of clause 7, further comprising comparing the state of synchrony to a predetermined threshold stored in memory.
Clause 9. The method of any of clauses 7-8, further comprising: determining, by the controller, an aggregate parameter based on the first neuromodulatory signal and the second neuromodulatory signal; comparing the aggregate parameter to a limit threshold; and ceasing further emission of neuromodulatory signals based on the comparison.
Clause 10. The method of clause 9, wherein the aggregate parameter comprises at least one parameter selected from the group consisting of total emission time and total emission power.
Clause 11. The method of any of clauses 1-10, further comprising storing the adjusted parameter in memory.
Clause 12. The method of any of clauses 1-11, wherein the first biomarker signal comprises at least one signal selected from the group consisting of an electroencephalogram signal, a magnetoencephalography signal, and a heart rate.
Clause 13. A system for inducing sleep in a subject, the system comprising: a transducer capable to emit a first neuromodulatory signal to the subject based on a signal parameter during a first time period; a detector capable to measure a biomarker signal of the subject during a second time period, wherein the second time period is subsequent to or at least partially overlaps with the first time period; and a controller in signal communication with the transducer and the detector, the controller capable to: determine a characteristic of the biomarker signal; adjust the signal parameter based on the at least one characteristic thereby forming an adjusted parameter; and based on the adjusted parameter, perform at least one of the following: cause the transducer to emit a second neuromodulatory signal to the subject during a third time period based on the adjusted parameter; or cease further emission of neuromodulatory signals based on the adjusted parameter.
Clause 14. The system of clause 13, wherein the transducer comprises at least one element selected from the group consisting of an electrode, an ultrasonic transducer, an electromagnetic field transducer, a visual transducer, and an acoustic transducer.
Clause 15. The system of any of clauses 13-14, wherein the detector comprises at least one element selected from the group consisting of an electroencephalogram detector, a magnetoencephalography detector, and a heart monitor.
Clause 16. The system of any of clauses 13-15, wherein the transducer comprises an electrode and the detector comprises an electroencephalogram detector.
Clause 17. The system of any of clauses 13-16, wherein the controller is capable to cause the transducer to emit a second neuromodulatory signal to the subject during a third time period based on the adjusted parameter.
Clause 18. The system of any of clauses 13-17, wherein the signal parameter comprises at least one parameter selected from the group consisting of time of stimulation, intensity of stimulation, frequency of stimulation, frequency of application of stimulation, rate of application of stimulation, and a state parameter.
Clause 19. The system of any of clauses 13-18, wherein the characteristic of the biomarker signal comprises at least one characteristic selected from the group consisting of a heart rate, a synchronous measurement, an amplitude based measurement, a delta band spectral power, a phase locking value, and a means squared coherence.
Clause 20. The system of any of clauses 13-19, further comprising a headband and wherein the transducer and the detector are disposed within or on the headband.

In the present disclosure, unless otherwise indicated, all numerical parameters are to be understood as being prefaced and modified in all instances by the term “about,” in which the numerical parameters possess the inherent variability characteristic of the underlying measurement techniques used to determine the numerical value of the parameter. At the very least, and not as an attempt to limit the application of the doctrine of equivalents to the scope of the claims, each numerical parameter described herein should at least be construed in light of the number of reported significant digits and by applying ordinary rounding techniques.

Also, any numerical range recited herein includes all sub-ranges subsumed within the recited range. For example, a range of “1 to 10” includes all sub-ranges between (and including) the recited minimum value of 1 and the recited maximum value of 10, that is, having a minimum value equal to or greater than 1 and a maximum value equal to or less than 10. Any maximum numerical limitation recited in this specification is intended to include all lower numerical limitations subsumed therein, and any minimum numerical limitation recited in the present disclosure is intended to include all higher numerical limitations subsumed therein. Accordingly, Applicant reserves the right to amend the present disclosure, including the claims, to expressly recite any sub-range subsumed within the ranges expressly recited. All such ranges are inherently described in the present disclosure.

The grammatical articles “a”, “an”, and “the”, as used herein, are intended to include “at least one” or “one or more”, unless otherwise indicated, even if “at least one” or “one or more” is expressly used in certain instances. Thus, the foregoing grammatical articles are used herein to refer to one or more than one (i.e., to “at least one”) of the particular identified elements. Further, the use of a singular noun includes the plural, and the use of a plural noun includes the singular, unless the context of the usage requires otherwise.

One skilled in the art will recognize that the system, systems, structures, methods, operations/actions, and objects described herein, and the discussion accompanying them, are used as examples for the sake of conceptual clarity and that various configuration modifications are contemplated. Consequently, as used herein, the specific examples/embodiments set forth and the accompanying discussion are intended to be representative of their more general classes. In general, use of any specific exemplar is intended to be representative of its class and the non-inclusion of specific components, devices, system, operations/actions, and objects should not be taken as limiting. While the present disclosure provides descriptions of various specific aspects for the purpose of illustrating various aspects of the present disclosure and/or its potential applications, it is understood that variations and modifications will occur to those skilled in the art. Accordingly, the invention or inventions described herein should be understood to be at least as broad as they are claimed and not as more narrowly defined by particular illustrative aspects provided herein.

What is claimed is:

1. A method for inducing sleep in a subject, the method comprising:
emitting, by a transducer, a first neuromodulatory signal to the subject based on a signal parameter during a first time period;
measuring, by a first detector, a first biomarker signal of the subject during a second time period, wherein the second time period is subsequent to or at least partially overlaps with the first time period;
determining, by a controller, a characteristic of the first biomarker signal;
adjusting, by the controller, the signal parameters based on the characteristic thereby forming an adjusted parameter; and
based on the adjusted parameter, performing at least one of the following:
emitting, by the transducer, a second neuromodulatory signal to the subject during a third time period based on the adjusted parameter; or
ceasing further emission of neuromodulatory signals based on the adjusted parameter.

2. The method of claim 1, comprising emitting, by the transducer, the second neuromodulatory signal to the subject during the third time period based on the at least one adjusted parameter.

3. The method of claim 1, wherein the first neuromodulatory signal comprises at least one form of non-invasive neuromodulation selected from the group consisting of an electrical stimulation, ultrasound stimulation a magnetic field stimulation, an ultrasound stimulation, a visual stimulation, and acoustic stimulation.

4. The method of claim 1, wherein the signal parameter comprises at least one parameter selected from the group consisting of time of stimulation, intensity of stimulation, frequency of stimulation, frequency of application of stimulation, rate of application of stimulation, and a state parameter.

5. The method of claim 1, wherein the first time period is in a range of 4 seconds to 8 seconds and the second time period is in a range of 1 second to 60 seconds.

6. The method of claim 1, wherein the characteristic of the first biomarker signal comprises at least one characteristic selected from the group consisting of a heart rate, a synchronous measurement, an amplitude based measurement, a delta band spectral power, a phase locking value, and a means squared coherence.

7. The method of claim 1, wherein the first biomarker signal is related to a first portion of a brain of the subject and the method further comprises:
measuring, by a second detector, a second biomarker signal of the subject during the second time period, wherein the second biomarker signal is related to a second portion of the brain of the subject different than the first portion; and
comparing the first biomarker signal and the second biomarker signal to determine a state of synchrony, wherein the characteristic comprises the state of synchrony.

8. The method of claim 7, further comprising comparing the state of synchrony to a predetermined threshold stored in memory.

9. The method of claim 7, further comprising:
determining, by the controller, an aggregate parameter based on the first neuromodulatory signal and the second neuormodulatory signal;
comparing the aggregate parameter to a limit threshold; and
ceasing further emission of neuromodulatory signals based on the comparison.

10. The method of claim 9, wherein the aggregate parameter comprises at least one parameter selected from the group consisting of total emission time and total emission power.

11. The method of claim 1, further comprising storing the adjusted parameter in memory.

12. The method of claim 1, wherein the first biomarker signal comprises at least one signal selected from the group consisting of an electroencephalogram signal, a magnetoencephalography signal, and a heart rate.

13. A system for inducing sleep in a subject, the system comprising:
a transducer capable to emit a first neuromodulatory signal to the subject based on a signal parameter during a first time period;
a detector capable to measure a biomarker signal of the subject during a second time period, wherein the second time period is subsequent to or at least partially overlaps with the first time period; and
a controller in signal communication with the transducer and the detector, the controller capable to:
determine a characteristic of the biomarker signal;
adjust the signal parameter based on the at least one characteristic thereby forming an adjusted parameter; and
based on the adjusted parameter, perform at least one of the following:
cause the transducer to emit a second neuromodulatory signal to the subject during a third time period based on the adjusted parameter; or
cease further emission of neuromodulatory signals based on the adjusted parameter.

14. The system of claim 13, wherein the transducer comprises at least one element selected from the group consisting of an electrode, an ultrasonic transducer, an electromagnetic field transducer, a visual transducer, and an acoustic transducer.

15. The system of claim 13, wherein the detector comprises at least one element selected from the group consisting of an electroencephalogram detector, a magnetoencephalography detector, and a heart monitor.

16. The system of claim 13, wherein the transducer comprises an electrode and the detector comprises an electroencephalogram detector.

17. The system of claim 13, wherein the controller is capable to cause the transducer to emit a second neuromodulatory signal to the subject during a third time period based on the adjusted parameter.

18. The system of claim 13, wherein the signal parameter comprises at least one parameter selected from the group consisting of time of stimulation, intensity of stimulation, frequency of stimulation, frequency of application of stimulation, rate of application of stimulation, and a state parameter.

19. The system of claim 13, wherein the characteristic of the biomarker signal comprises at least one characteristic selected from the group consisting of a heart rate, a synchronous measurement, an amplitude based measurement, a delta band spectral power, a phase locking value, and a means squared coherence.

20. The system of claim 13, further comprising a headband and wherein the transducer and the detector are disposed within or on the headband.
